SEQUENCE LISTING (5 sequences)

SEQ ID NO: 1 - protein, 541 aa, Artificial Sequence
  RTA-PAPS1
MIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNINTITFDAGNATINKYATFMESLRNEAKDPSLKCYGIPMLPNTNSTIKYLLVKLQGASLKTITLMLRRNNLYVMGYSDPYDNKCRYHIFNDIKGTEYSDVENTLCPSSNPRVAKPINYNGLYPTLEKKAGVTSRNQVQLGIQILSSDIGKISGQGSFTEKIEAKFLLVAIQMVSEAARFKYIENQVKTNFNRDFSPNDKVLDLEENWGKISTAIHNSKNGALPKPLELKNADGTKWIVLRVDEIKPDVGLLNYVNGTCQAT

SEQ ID NO: 2 - protein, 556 aa, Artificial Sequence
  RTAM-PAPS1
MHHHHHHIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIIAIQMISEAARFQYIEGEMRTRIRYNRRSAPDPSVITLENSWGRLSTAIQESNQGAFASPIQLQRRNGSKFSVYDVSILIPIIALMVYRAAPPPSSQFGGGGSDVADIGGGGSGGGGSVNTIIYNVGSTTISKYATFLNDLRNEAKDPSLKCYGIPMLPNTNTNPKYVLVELQGSNKKTITLMLRRNNLYVMGYSDPFETNKCRYHIFNDISGTERQDVETTLCPNANSRVSKNINFDSRYPTLESKAGVKSRSQVQLGIQILDSNIGKISGVMSFTEKTEAEFLLVAIQMVSEAARFKYIENQVKTNFNRAFNPNPKVLNLQETWGKISTAIHDAKNGVLPKPLELVDASGAKWIVLRVDEIKPDVALLNYVGGSCQTT

SEQ ID NO: 3 - DNA, 48 bp, Artificial Sequence
  primer RP1-A48
tttaactttaagaaggagatatacatatgatcttcccgaaacagtacc

SEQ ID NO: 4 - DNA, 48 bp, Artificial Sequence
  primer RPAP1-A48
tttaactttaagaaggagatatacatatgcaccaccatcaccaccata

SEQ ID NO: 5 - DNA, 50 bp, Artificial Sequence
  primer RPAP1-B50
cagccggatctcagtggtggtgctcgagttaggtagtctggcaagaaccg

(19) United States
(12) Patent Application Publication
(10) Pub. No.: US 2020/0392192 A1
(43) Pub. Date: Dec. 17, 2020

(54) ANTI-VIRAL PROTEINS

(71) Applicant: OPHIUCHUS MEDICINE INC, Vancouver (CA)

(72) Inventors: Yasser Salim HASSAN, St-Paul (FR); Sherry OGG, Silver Spring, MD (US)

(21) Appl. No.: 16/971,020

(22) PCT Filed: Mar. 29, 2019
(86) PCT No.: PCT/CA2019/050388

§ 371 Date: Aug. 19, 2020

Related U.S. Application Data
(60) Provisional application No. 62/661,836, filed on Apr. 24, 2018.

(51) Int. Cl. C07K 14/415; A61K 47/65; A61P 31/20

(52) U.S. Cl. CPC C07K 14/415; A61P 31/20; A61K 47/65

(57) ABSTRACT

It is provided an anti-viral fusion protein comprising the structure of X-Y-Z, wherein X is a full length Ricin A chain (RTA) or a variant thereof, Y is absent or a linker and Z is a full length Pokeweed antiviral proteins (PAP) or a variant thereof. Particularly, it is provided an optimized protein ricin A chain mutant-Pokeweed antiviral protein isoform 1 from leaves (RTAM-PAP1).

### CROSS-REFERENCE TO RELATED APPLICATIONS

The present application claims benefit of U.S. Provisional Application No. 62/661,836 filed Apr. 24, 2018, the content of which is hereby incorporated by reference in its entirety.

### TECHNICAL FIELD

It is provided an anti-viral fusion protein of ricin A chain protein (RTA) and the Pokeweed antiviral proteins (PAPs).

### BACKGROUND

Pokeweed antiviral proteins (PAPs) are expressed in several organs of the plant pokeweed (Phytolacca Americana) and are potent type I Ribosome Inactivating Proteins (RIPs). Their sizes vary from 29-kDa to 30-kDa and are able to inhibit translation by catalytically removing specific adenine residues from the large rRNA of the 60S subunit of eukaryotic ribosomes. Furthermore, PAPs can depurinate specific guanine residues, in addition to adenine, from the rRNA of prokaryotic ribosomes. PAPs possess antiviral activity on a wide range of plant and human viruses through various mechanisms. Transgenic plants expressing different forms of PAPs were found to be resistant to various viral and fungal infections. The anti-viral activity of PAPs against human viruses has been described against Japanese encephalitis virus (Ishag et al., 2013, Virus Res., 171: 89-96), human immunodeficiency virus-1 (HIV-1) (Rajamohan et al., 1999, Biochem Biophys Res Commun., 260: 453-458), human T-cell leukemia virus-1 (HTLV-1) (Mansouri et al., 2009, J Biol Chem., 284: 31453-31462), herpes simplex virus (HSV) (Aron and Irvin, 1980, Antimicrob Agents Chemother., 17: 1032-1033), influenza (Tomlinson et al., 1974, J. Gen. Virol., 22: 225-232), hepatitis B virus (HBV) (He et al., 2008, World J Gastroenterol., 14: 1592-1597), and poliovirus (Ussery et al., 1977, Ann N Y Acad Sci., 284: 431-440).

Ricin is expressed in the seeds of the castor oil plant (Ricinus communis) and is one of the most potent type II RIPs. It is highly toxic to mammalian cells as its A chain can efficiently be delivered into the cytosol of cells through the mechanism of its B chain. The B chain serves as a galactose/N-acetylgalactosamine binding domain (lectin) and is linked to the A chain via disulfide bonds. Ricin can induce 50% apoptosis in mammalian cells at concentrations below 1 ng/mL while showing no to low activity on plant and E. coli ribosomes. The ricin A chain (RTA) on its own has less than 0.01% of the toxicity of the native protein in a cell culture test system. It was furthermore shown that RTA alone had no activity on non-infected and tobacco mosaic virus (TMV)-infected tobacco protoplasts alike. RTA lacks the ability to enter the cell without the action of the B chain. RTA depurinates a universally conserved adenine residue within the sarcin/ricin loop (SRL) of the 28S rRNA to inhibit protein synthesis. Though there are currently no commercially available therapeutic applications, RTA is extensively studied in the development of immunotoxins.

The therapeutic potential of PAPs and RTA has been explored for over thirty years, though dosage dependent side effects have limited clinical applications. These proteins have shown very low cytotoxicity to non-infected cells; however, PAPs administration in mouse models has resulted in hepatic, renal and gastrointestinal tract damage with a median lethal dose (LD50) as low as 1.6 mg/Kg (Benigni et al., 1995, Int J Immunopharmacol., 17: 829-839). Interestingly, RTA shows no toxicity even at high doses with similar half-life times. Nevertheless, all RIPs show immunosuppressive effects to various degrees. Many studies have described the various dose-limiting side effects of these proteins when used as immunotoxins (i.e. vascular leak syndrome, hemolytic uremic syndrome and pluritis, among others) (Schindler et al., 2011, British Journal of Haematology, 154: 471-476; Meany et al., 2015, Journal of immunotherapy, 38: 299-305).

The engineering of novel therapeutic fusion proteins with higher specificity, selectivity, and potency with fewer side effects is a leading strategy in drug development that is more often than not limited by current understanding of protein structure and function. Another limiting factor is the availability of efficient protein structure prediction and simulation software.

There is still a need to be provided with new molecules acting against infectious diseases and that will be cheaper to produce than available therapeutics.

### SUMMARY

It is provided an anti-viral fusion protein comprising the structure:

X-Y-Z

wherein X is a full length Ricin A chain (RTA) or a variant thereof, Y is absent or a linker and Z is a full length Pokeweed antiviral protein (PAP) or a variant thereof.

In an embodiment, Z is the Pokeweed Antiviral Protein from Leaves (PAP1).

In another embodiment, PAP1 comprises amino acids 296-556 of SEQ ID NO: 2.

In an embodiment, the linker is chemical linker or a polylinker.

In a further embodiment, the linker is a flexible linker.

In another embodiment, the flexible linker comprises amino acids 275-295 of SEQ ID NO: 2.

In an additional embodiment, X is a mutant of RTA (RTAM).

In an embodiment, RTAM comprises amino acids 8-274 of SEQ ID NO: 2.

In an embodiment, the fusion protein described herein comprises the amino acid sequence of SEQ ID NO: 1.

In an embodiment, the fusion protein described herein comprises the amino acid sequence of SEQ ID NO: 2.

In an embodiment, the fusion protein described herein is for treating a viral infection.

In an embodiment, the viral infection is from the Hepatitis B virus (HBV), Hepatitis C virus (HCV), Kaposi Sarcoma-Associated Herpesvirus (KSHV), Merkel Cell Polyomavirus (MCV). Human T-Cell Lymphotropic Virus Type 1 (HTLV-1), Epstein-Barr Virus (EBV), human immunodeficiency virus-1 (HIV-1), Zika virus, Japanese encephalitis virus, Herpes Simplex, Poliovirus, Influenza virus or papillomavirus.

In another embodiment, the viral infection causes liver cancer, Kaposi sarcoma, skin cancer, Merkel cell carcinoma, leukemia, lymphoma, Burkitt's lymphoma, Nasopharyngeal carcinoma, Hodgkin's lymphoma, non-Hodgkin's lymphoma, T-cell lymphomas, Post-transplant lymphoproliferative disorder, or Leiomyosarcoma.

In a further embodiment, the viral infection is from HBV.

In another embodiment, the viral infection is from Zika virus.

In an embodiment, the fusion protein described herein is active against plant, animal or human pathogens.

It is also provided a fusion protein comprising the amino acid sequence of SEQ ID NO: 2.

It is further provided a composition comprising the fusion protein as described herein and a carrier.

It is further provided a method of treating a viral infection in a patient comprising administering to the patient the fusion protein described herein.

It is additionally provided the use of the fusion protein described herein for treating a viral infection in a patient.

### BRIEF DESCRIPTION OF THE DRAWINGS

Reference will now be made to the accompanying drawings.

FIG. 1 illustrates the medium optimization and protein purification showing in (A) medium optimization for Ricin-PAPS1 (RP1) expression, wherein three different growth media including M9 (M9), Luria Bertani (LB) and terrific broth (TB) were tested for Ricin-PAPS1 expression at 30° C., soluble lysate (Sol) and inclusion body (IB) from each sample were analyzed by SDS PAGE and visualized by Coomassie blue staining; and in (B) validation of purified Ricin-PAPS1 protein, wherein recombinant Ricin-PAPS1 was produced in 1 L of culture that was induced with the optimized condition (LB medium with 1 mM IPTG at 30° C. for 4 hrs) and purified from inclusion bodies through gel filtration before refolding, concentration and dialysis, the resulting protein of approx. 60.5 kDa was >90% purity determined by SDS-PAGE.

FIG. 2 illustrates a test of purified RTA-PAPS1 in the TnT transcription/translation assay, wherein five different concentration points (0.01 nM, 0.02 nM, 0.03 nM, 0.08 nM, 0.25 nM) were examined, values are calculated as percent Luciferase protein synthesis compared to control, and results represent the mean for two individual experiments and the curve is the logarithmic regression (Std Error=Std Deviation/(SQRT(n)), with n=2).

FIG. 3 is an anti-HBV evaluation of RTA-PAPS1, wherein recombinant RTA-PAPS1 was tested for its anti-HBV activity using 6 concentrations using a serial dilution by a factor of 10 in growth media (600 nM, 60 nM, 6 nM, 0.6 nM, 0.06 nM, 0.006 nM for RTA-PAPS1 and 10000 nM, 1000 nM, 100 nM, 10 nM, 1 nM, 0.1 nM for 3TC), and values are calculated as percent of virus DNA control [(amount of virus DNA in treated sample/amount of virus DNA in untreated sample)×100], results representing the mean for three individual experiments (Std Error=Std Deviation/(SQRT(n)), with n=3).

FIG. 4 illustrates the predicted 3D Protein Structure, showing in (A) protein structure as determined by Phyre2 with the arrows showing the flexible linker at position 275-294 and the CASP2 recognition site at position 280-284; and in (B) the ligand binding sites of RTAM moiety (up) and of PAP1 moiety (down) as determined by I-Tasser (using the Phyre2 model as one of the templates).

FIG. 5 illustrates the production and purification of native RTAM-PAP1, showing in (A) loosely bound proteins were washed with the lysis buffer containing 50 mM imidazole (I₅₀) on a Ni-sepharose column and RTAM-PAP1 (RPAP1) proteins were then eluted with the elution buffer containing 300 mM Imidazole (1300); in (B) the Western Blot using ricin a chain antibody RA999 confirmed the presence of RTAM-PAPS1 at approx. 61.5 kDa, wherein the bands between 21 kDa and 32 kDa are assumed to be degraded or/and premature RTAM-PAP1 proteins; in (C) (Lys) from 1 L culture; in (D) co-purified host cell proteins were further separated by a hydroxylapatite column, wherein most RTAM-PAP1 proteins were retained in the flow through (FT) fraction, while most host cell proteins were bound to the hydroxylapatite column (P200 elution); in (E) RTAM-PAP1 was peaked at fraction 15 and 16, the purest fraction (F15) was estimated at >95% homogeneity; and in (F) the inhibition assay.

FIG. 6 illustrates comparative inhibition activity of RTAM-PAP1 and RTA-PAPS1 in the TnT transcription/translation assay; wherein five different concentration points (0.01 nM, 0.02 nM, 0.03 nM, 0.08 nM, 0.25 nM for RTA-PAPS1 and 0.02 nM, 0.03 nM, 0.06 nM, 0.16 nM, 0.40 nM for RTAM-PAP1) were examined, values are calculated as percent Luciferase protein synthesis compared to control, and results representing the mean for two individual experiments and the curves are the logarithmic regression for RTA-PAPS1 and power regression for RTAM-PAP1 ((Std Error=Std Deviation/(SQRT(n)), with n=2).

### DETAILED DESCRIPTION

It is provided an anti-viral fusion protein of ricin A chain protein (RTA) and the Pokeweed antiviral proteins (PAPs).

Ricin A chain (RTA) and Pokeweed antiviral proteins (PAPs) are plant-derived N-glycosidase ribosomal-inactivating proteins (RIPs) isolated from Ricinus communis and Phytolacca Americana respectively. It is provided herein the amenability and sub-toxic antiviral value of a novel fusion protein between RTA and PAPs (RTA-PAPs). RTA-Pokeweed antiviral protein isoform 1 from seeds (RTA-PAPS1; previously described in WO2017/175060, the content of which is incorporated herein in its entirety) was produced in an E. coli in vivo expression system, purified from inclusion bodies using gel filtration chromatography and protein synthesis inhibitory activity assayed by comparison to the production of a control protein Luciferase. The antiviral activity of the RTA-PAPS1 against Hepatitis B virus (HBV) in HepAD38 cells was then determined using a dose response assay by quantifying supernatant HBV DNA compared to control virus infected HepAD38 cells. The cytotoxicity in HepAD38 cells was determined by measuring cell viability using a tetrazolium dye uptake assay. The fusion protein was further optimized using in silico tools, produced in an E. coli in vivo expression system, purified by a three-step process from soluble lysate and confirmed in a protein synthesis inhibition activity assay.

Fusion and hybrid proteins of RTA and PAPs have also been developed in pursuit of selectively targeting infected cells and selectively recognizing viral components, though with limited success (Rothan et al., 2014, Antiviral Res., 108, 173-180; Chaddock et al., 1996, Eur J Biochem., 235: 159-166).

Based on the data gathered on these two proteins over the last thirty years and the newly available in silico tools, it is described herein the creation of a novel fusion protein between RTA and PAPs that is more effective than either of the proteins alone at sub-toxic dosages against specific infectious diseases and that is cheaper to produce than available therapeutics.

It is provided herein an effective and scalable production system in Escherichia coli and of purification methods that enabled accurate determination of RTA-PAPs protein synthesis inhibition in vitro. The in vitro reduced cytotoxicity and significant anti-HBV activity of RTA-Pokeweed antiviral protein isoform 1 from seeds (RTA-PAPS1) is described by detecting HBV DNA in the supernatant of HepAD38 cells. The reengineering of RTA-PAPS1 into RTA mutant-Pokeweed antiviral protein isoform 1 from leaves (RTAM-PAP1) to improve its production in Escherichia coli and to enhance its gain of function is also described using the most up-to-date protein structure and function prediction software available online.

As used herein, the term “RIP” refers to ribosome inactivating proteins. As used herein, the terms “PAP” or “pokeweed antiviral protein” refer to a polypeptide with substantial or complete sequence homology to pokeweed antiviral protein or a polynucleotide encoding such a polypeptide, which may or may not include a signal peptide as evident by the context in which the term is used (for example, GenBank Entry Accession No. KT630652). When no variant is specified, PAP may refer to the unmodified polypeptide or polynucleotide or to a variant of PAP. As used herein, the terms “RTA” or “ricin A-chain” refer to a polypeptide or a polynucleotide encoding a polypeptide with substantial or complete sequence homology to ricin A-chain GenBank Entry Accession No. X52908.

It is demonstrated that RTA-PAPS1 could effectively be recovered and purified from inclusion bodies. The refolded protein was bioactive with a 50% protein synthesis inhibitory concentration (IC₅₀) of 0.06 nM (3.63 ng/ml). RTA-PAPS1 has a synergetic activity against HBV with a half-maximal response concentration value (EC₅₀) of 0.03 nM (1.82 ng/ml) and a therapeutic index of >21818 with noticeable steric hindrance. The optimized protein ricin A chain mutant-Pokeweed antiviral protein isoform 1 from leaves (RTAM-PAP1) can be recovered and purified from soluble lysates with gain of function on protein synthesis inhibition activity, with an IC₅₀ of 0.03 nM (1.82 ng/ml), and with minimal, if any, steric hindrance.

RTA-PAPS1 is a monomeric polypeptide of 541 amino acids with an apparent molecular mass of 60.5 kDa, with the following amino acid sequence: MIFPKQYPIINFTTAGATVQSYTNFIRAVRGRLTTGADVRHEIPVLPNRVGLPINQRFILVELS NHAELSVTLALDVTNAYVVGYRAGNSAYFFHPDNQEDAEAITHLFTDVQNRYTFAFGGNYDRLE QLAGNLRENIELGNGPLEEAISALYYYSTGGTQLPTLARSFIICIQMISEAARFQYIEGEMRTR IRYNRRSAPDPSVITLENSWGRLSTAIQESNQGAFASPIQLQRRNGSKFSVYDVSILIPIIALM VYRCAPPPSSQFSLLIRPVVPNFNINTITFDAGNATINKYATFMESLRNEAKDPSLKCYGIPML PNTNSTIKYLLVKLQGASLKTITLMLRRNNLYVMGYSDPYDNKCRYHIFNDIKGTEYSDVENTL CPSSNPRVAKPINYNGLYPTLEKKAGVTSRNQVQLGIQILSSDIGKISGQGSFTEKIEAKFLLV AIQMVSEAARFKYIENQVKTNFNRDFSPNDKVLDLEENWGKISTAIHNSKNGALPKPLELKNAD GTKWIVLRVDEIKPDVGLLNYVNGTCQAT (SEQ ID NO: 1).

RTA-PAPs are amenable to effective production and purification in native form, possess significant gain of function on protein synthesis inhibition and anti-HBV activities in vitro with a high therapeutic index and, thus, is a potent antiviral agent against chronic HBV infection to be used as a standalone or in combination with existent therapies.

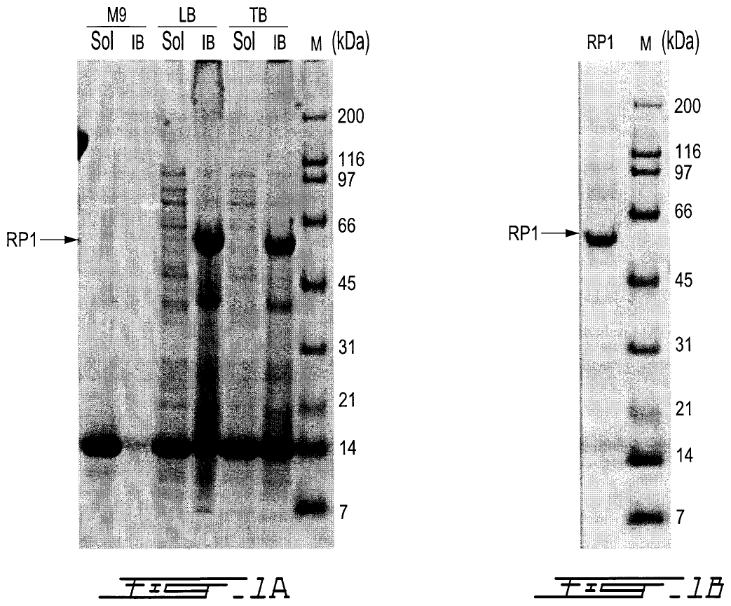

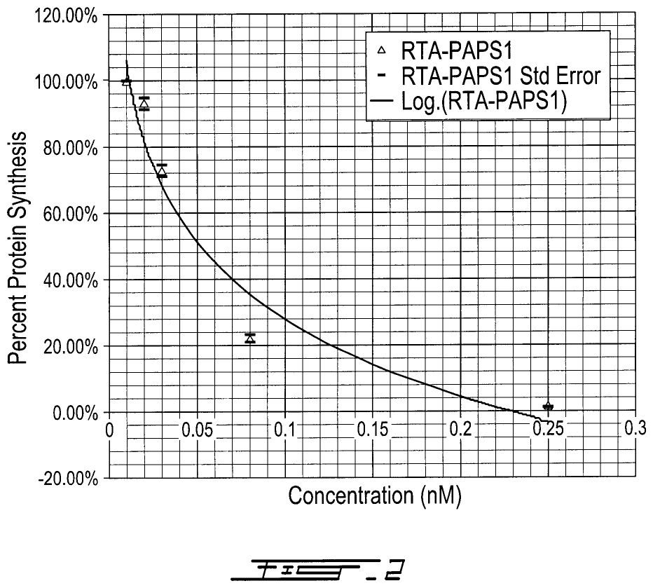

The production of fusion Ricin A Chain-Pokeweed Antiviral Protein from Seeds Isoform 1 (RTA-PAPS1) in E. coli was found to be significantly better at 30° C. than at 37° C. In order to optimize the amount of protein produced from 1 L at 30° C., three media were tested: M9 (M9), Luria Bertani (LB) and terrific broth (TB). Soluble lysate (Sol) and inclusion body (IB) from each sample were analyzed by SDS PAGE and visualized by Coomassie blue staining (FIG. 1A). As can be seen, almost all of the overexpressed RTA-PAPS1 proteins were in the form of inclusion bodies, which were almost completely insoluble in either 6M Urea or 6M Guanidine. A total of 28 proprietary buffers were tested and only the denaturing buffer 8b (proprietary formulation of AscentGene) was able to dissolve more than 50% of the Ricin-PAPS1 present in the inclusion bodies. Once the soluble proteins were recovered and purified through the gel filtration column Superdex200 (single step) in their denatured form, they were allowed to refold for over 20 hrs in a refolding buffer before being concentrated. The resulting protein was found to be at a concentration of 0.22 mg/ml at >90% purity (FIG. 1B).

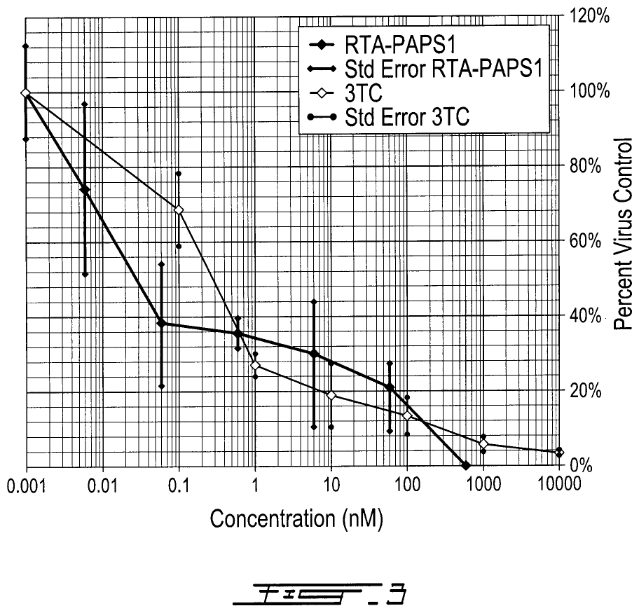

The inhibitory activity of RTA-PAPS1 was determined using 5 different concentrations of purified RTA-PAPS1 in duplicate with the Rabbit Reticulate Lysate TnT® system using Luciferase as control. A Luciferase assay was used to determine Luciferase expression levels using a luminometer. The resulting plot is shown in FIG. 2 while taking the standard deviation into account. As can be observed, the difference between the duplicate results is very minimal. The standard deviation varied from 0.10% to 5% leading to very small standard errors. It can further be observed that RTA-PAPS1 has an IC₅₀ at 0.06 nM, slower than RTA IC₅₀ at 0.03 nM but comparable to PAPS IC₅₀ at 0.07. The IC₁₀₀ however is attained faster than any of them at 0.24 nM for RTA-PAPS1, twice as fast as RTA IC₁₀₀ at 0.60 nM. These results show that RTA-PAPS1 is bioactive with a synergetic activity between the RTA and PAPS1 moieties being noticeable.

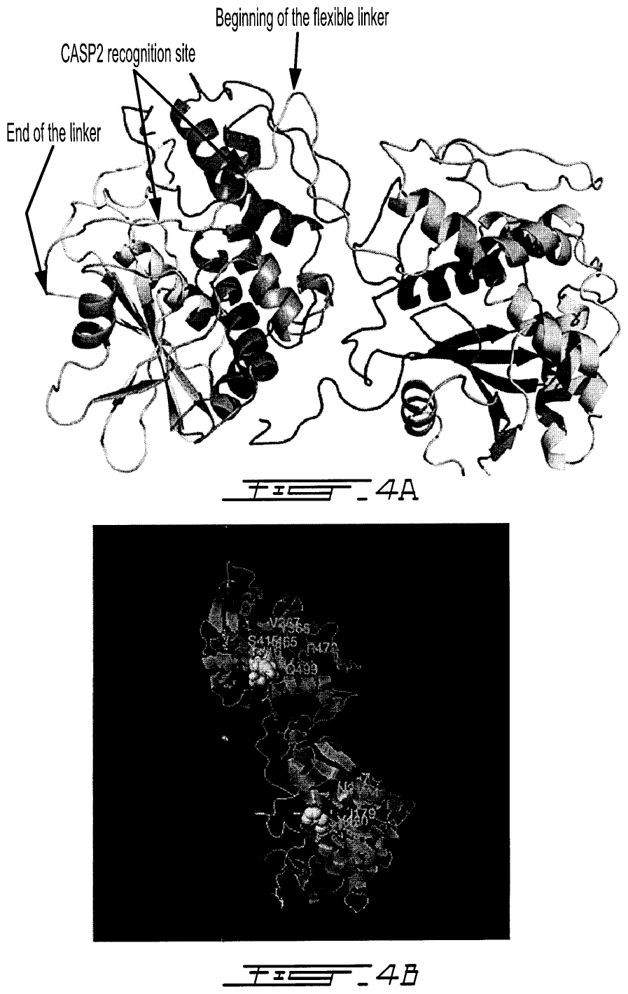

Recombinant RTA-PAPS1 was evaluated for anti-HBV activity and cytotoxicity in the HBV chronically infected cell line AD38 using a six concentrations dose response assay in triplicate. The lamivudine (3TC) control compound was evaluated in parallel. The antiviral efficacy based on quantified DNA copies in the supernatant of both compounds are shown in FIG. 3 in a plot form. RTA-PAPS1 yielded a half-maximal response concentration value (EC₅₀) of 0.03 nM while 3TC yielded an EC₅₀ of 0.3 nM, which is a ten-fold difference. RTA-PAPS1 was not cytotoxic to HepAD38 cells at concentrations up to 600 nM. These results led to a therapeutic index for RTA-PAPS1 of >21818, which is a huge improvement over values given in the literature (EC₅₀ of 330 nM and a therapeutic index of 9.3 for PAPS1 alone under comparable conditions on HepG2 2.2.15 cells) (He et al., 2008, World J Gastroenterol, 14: 1592-1597). These results clearly show the significant anti-HBV activity of RTA-PAPS1.

RTA-PAPS1 was found to be very effective against Hepatitis B Virus and also effective on HIV1, Zika and Hepatitis C Virus as shown. In anti-viral cytoprotection assay, as provided in Tables 1-4, RTA-PAPS1 showed high Therapeutic Index (TI) for HBV, which is preferable for a drug to have a favorable safety and efficacy profile, and high efficacy for HIV1, Zika and HCV.

TABLE 1
Anti-HIV1 cytoprotection assay

| | CEM-SS/HIV_{RF} | | |
|---|---|---|---|
| Compound | EC₅₀ (μM) | TC₅₀ (μM) | TI |
| RTA-PAPS1 | 0.19 | >0.6 | >3.16 |
| AZT | 0.0008 | >1 | >1250 |

TABLE 2
Anti-Zika cytoprotection assay

| | HUH7-Zika_{PRVABC59} | | |
|---|---|---|---|
| Compound | EC₅₀ (μM) | TC₅₀ (μM) | TI |
| RTA-PAPS1 | 0.05 | 0.06 | 1.2 |
| Sofosbuvir | 2.09 | >10 | >4.78 |

TABLE 3
Anti-HCV cytoprotection assay

| | HCV Replicon | | |
|---|---|---|---|
| Compound | EC₅₀ (μM) | TC₅₀ (μM) | TI |
| RTA-PAPS1 | 0.012 | 0.04 | 3.42 |
| Sofosbuvir | 0.05 | >1 | >18.5 |

TABLE 4
Anti-HBV cytoprotection assay

| | HBV AD38 | | |
|---|---|---|---|
| Compound | EC₅₀ (μM) | TC₅₀ (μM) | TI |
| RTA-PAPS1 | 0.00003 | >0.6 | >21818 |
| 3TC | 0.0003 | >10 | >35714 |

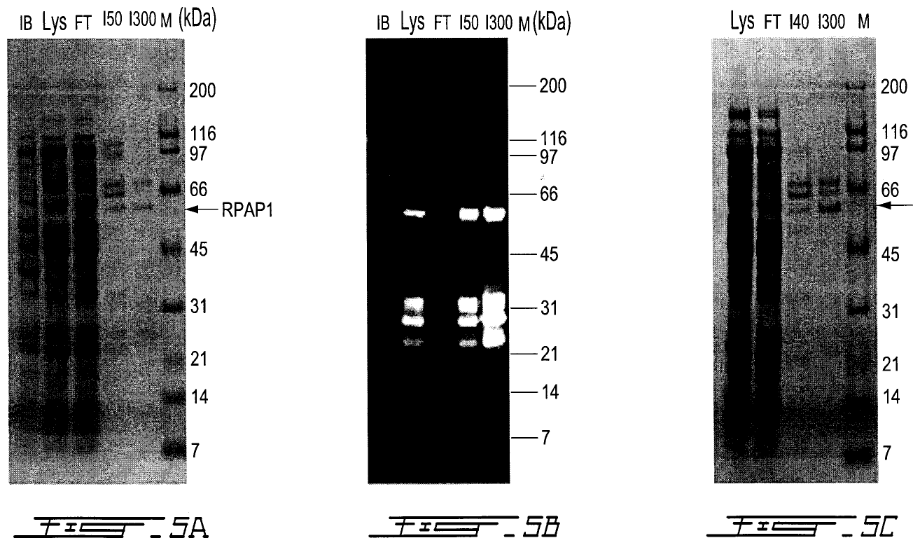

The design of the recombinant protein RTA-PAPS1 was completely revisited in order to further enhance the effect of the chimeric protein on HBV, reduce general toxicity and increase solubility to improve expression. The resulting design Ricin A Chain Mutant-Pokeweed Antiviral Protein from Leaves (RTAM-PAP1) was run through I-Tasser and Phyre2 and the resulting 3D models validated by Verify 3D. The model generated by Phyre2 passed Verify 3D while the one generated by I-Tasser failed. The one generated by Phyre2 was thus chosen as one of the templates to run I-Tasser again. The newly generated structure by I-Tasser scored higher on Verify 3D than the one generated by Phyre2 and was thus chosen as the model for the other software. The proper disulfide bond formations were confirmed by the DiANNA 1.1 webserver (at positions 328-553 and 379-400). The new model had a normalized QMEAN4 score of >0.6 and the introduction of the rigid CASP2 recognition site into the flexible linker at position 280-285 insured safe distance between the two proteins to safeguard the function of both moieties and minimize steric hindrance as can be seen in FIG. 4. The grand average of hydropathicity was reduced from −0.236 for RTA-PAPS1 to −0.265 for RTAM-PAP1 as was determined by ProtParam, which represents an improvement of 12% in hydrophilicity.

The anti-viral fusion protein RTAM-PAPS1 described herein comprises the following sequence:

(SEQ ID NO: 2)
MHHHHHHIFPKQYPIINFTTAGATVQSYTNFIRAVRGRLTTGADVRHEIP
VLPNRVGLPINQRFILVELSNHAELSVTLALDVTNAYVVGYRAGNSAYFF
HPDNQEDAEAITHLFTDVQNRYTFAFGGNYDRLEQLAGNLRENIELGNGP
LEEAISALYYYSTGGTQLPTLARSFIIAIQMISEAARFQYIEGEMRTRIR
YNRRSAPDPSVITLENSWGRLSTAIQESNQGAFASPIQLQRRNGSKFSVY
DVSILIPIIALMVYRAAPPPSSQFGGGGSDVADIGGGGSGGGGSVNTIIY
NVGSTTISKYATFLNDLRNEAKDPSLKCYGIPMLPNTNTNPKYVLVELQG
SNKKTITLMLRRNNLYVMGYSDPFETNKCRYHIFNDISGTERQDVETTLC
PNANSRVSKNINFDSRYPTLESKAGVKSRSQVQLGIQILDSNIGKISGVM
SFTEKTEAEFLLVAIQMVSEAARFKYIENQVKTNFNRAFNPNPKVLNLQE
TWGKISTAIHDAKNGVLPKPLELVDASGAKWIVLRVDEIKPDVALLNYVG
GSCQTT,

wherein amino Acids:

| | | |
|---|---|---|
| | 1 | Vector Starting Residue; |
| amino Acids: | 2-7 | 6-His Tag; |
| amino Acids: | 8-274 | Ricin A Chain (RTA); |
| amino Acids: | 275-295 | Flexible Linker + Casp2 Site; and |
| amino Acids: | 296-556 | Pokeweed Protein (PAP1). |

Accordingly, it is provided a fusion protein comprising the structure X-Y-Z, wherein X is the full length RTA or a variant thereof, Y is absent or a linker and Z is the full length PAP or a variant thereof. In an embodiment, X is RTA mutant (RTAM). In another embodiment, Z is the Pokeweed Antiviral Protein from Leaves (PAP1) as described herein.

The linker encompassed herein can be a chemical linker and/or a polylinker. Preferably, the linker is a flexible linker, i.e. composed of flexible residues like glycine and serine so that the adjacent protein domains are free to move relative to one another. A “chemical linker” as used herein is defined as a flexible linker, within some embodiments, the linker is a heterobifunctional linker, in some embodiments, the linker comprises a maleimido group. In various embodiments, the linker is selected from the group consisting of: GMBS; EMCS; SMPH; SPDP; and LC-SPDP.

The term “polylinker” or “linker peptide” as used herein is defined as a short segment of DNA added between the DNA encoding the fused proteins, to produce a short peptide or polypeptide to make it more likely that the proteins fold independently and behave as expected. This “polylinker” or “linker peptide” can also have cleavage sites for proteases or chemical agents that enable the liberation of the two separate proteins.

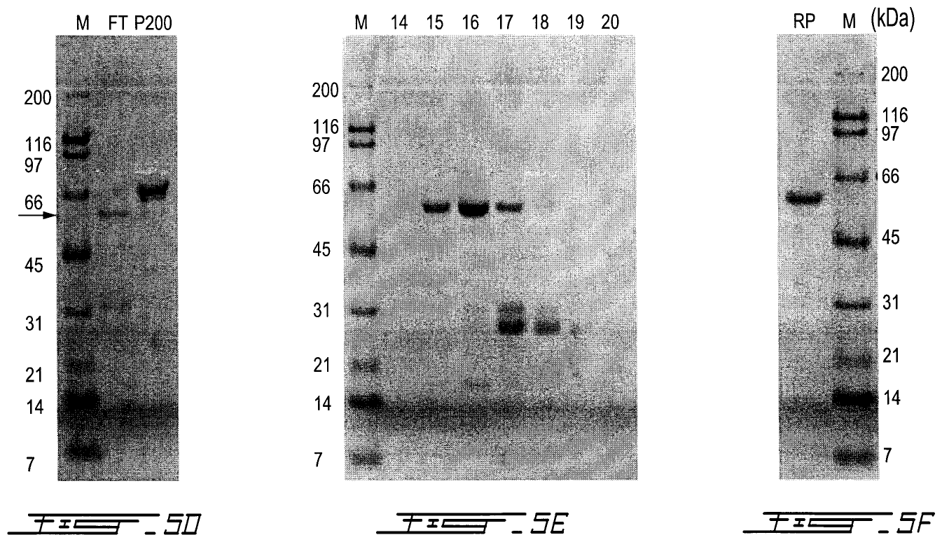

The production of RTAM-PAP1 was first tested under the same conditions as previously determined for RTA-PAPS1 and resulted in good production of native proteins. Soluble RTAM-PAP1 was recovered from the lysate, purified by Ni-sepharose column and analyzed by SDS-PAGE and Western Blot (FIGS. 5A and B). The production from 1 L culture under the same conditions gave equally good results (FIG. 5C). The purified proteins were then submitted to a second purification step using hydroxylapatite column, which showed good separation of RTAM-PAP1 from co-purified host proteins (FIG. 5D). The degraded (and/or premature) products were further separated by gel filtration on an FPLC column of Superose 12 (FIG. 5E) and the purest fraction (F15) reached >95% homogeneity at a concentration of 0.1 mg/ml (FIG. 5F) and was used for the protein synthesis inhibition assay.

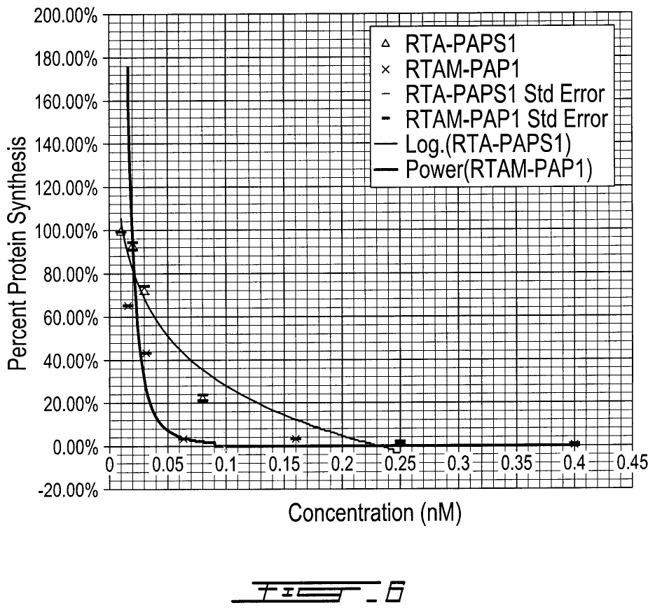

The inhibitory activity of RTAM-PAP1 was determined using 5 different concentrations, in duplicate, of purified RTAM-PAP1 on the Rabbit Reticulate Lysate TnT® system using Luciferase as the control as previously described. The resulting comparative plot of the activity on protein synthesis of both fusion proteins is shown in FIG. 6 while taking into account the standard deviations that ranged from 0.1% to 1%. As can be observed, the plot showed minimal difference between duplicates. It also shows that RTAM-PAP1 has an IC₅₀ at 0.03 nM, the same as RTA 10₅₀ at 0.03 nM, which is twice as fast as RTA-PAPS1 IC50 at 0.06 nM and about ten times faster than PAP1 IC₅₀ at 0.29 nM (Poyet et al., 1997, FEBS Lett., 406: 97-100). The IC₁₀₀ however is attained faster than any of them at 0.09 nM for RTAM-PAP1, which is a bit less than three times faster than RTA-PAPS1 IC₁₀₀ at 0.24 nM. These results show that RTAM-PAP1 is bioactive, both moieties' complementary catalytic activities functional, with minimal steric hindrance if any, and with a significant gain of function.

The chimeric protein RTA-PAPS1 was expressed only in inclusion bodies with very little solubility, except under heavy denaturing conditions. The refolding process was successful as more than one conformation was observed. This was probably due to the two free Cysteine residues in RTA and to the nature of the semi-flexible linker, which allowed the close proximity of Cys at position 260 to the Cys residues at position 364 and 385 (confirmed by DiANNA 1.1 webserver and I-Tasser). The addition of TCEP was necessary and a difference in bioactivity (>2 fold) was observed between samples. RTA-PAPS1 with the addition of TCEP was very bioactive and with a noticeable synergetic activity between RTA and PAPS1, which was probably limited by steric hindrance once again due to the nature of the semi-flexible quality of the linker. This was confirmed during the anti-HBV assays. The significant anti-HBV activity of RTA-PAPS1 was apparent and due to the ability of both moieties to depurinate rRNA but also polynucleotide, single-stranded DNA, double stranded DNA and mRNA. HBV is a double stranded DNA reverse transcriptase virus.

The fusion protein RTAM-PAP1 expression went very well as native protein production with high solubility was obtained (barely any in inclusions bodies). A three step purification protocol was in order to obtain soluble proteins with >90% homogeneity. Nonetheless, 0.1 mg of protein at >95% purity and 0.22 mg of protein at >90% purity were obtained from 1 L of culture. This yield is probably explained by the increased toxicity of PAP1 to E. coli compared to that of PAPS1 (>10 fold). The bioactivity of RTAM-PAP1 was increased, much more than expected with very little to no sign of steric hindrance. The introduction of the two point mutations in an embodiment in the RTA moiety and of the flexible linker further made a difference in solubility and activity. Also, perhaps, fine-tuning the formulation buffer to better preserve protein integrity allowed for optimum activity. The synergetic effect of both moieties was very apparent and due to the fact that RTA and PAP1 do not dock onto the ribosome at the same site and, thus, led to a reduction of partially depurinated and still functional ribosomes.

The chimeric proteins combining RTA and PAPs are potent novel broad range anti-viral proteins with gain of function in protein synthesis inhibition activity and anti-HBV activity in vitro with minimal cytotoxicity. As encompassed herein, the anti-viral proteins described herein have a broader anti-viral activity against plant, animal and human pathogens, including as trait in transgenic plants expressing it, as a stand-alone administration (therapeutics). In an embodiment, the broad range anti-viral proteins described herein are effective, for example and not limited to, against Group IV viruses (ssRNA viruses), Group V viruses (ssRNA viruses) and/or Group VI viruses (or ssRNA-RT viruses). The introduction of two point mutations in RTA and of a flexible linker further greatly improved solubility and activity. RTAM-PAP1 can be overexpressed, recovered and purified from soluble lysate. It is expected that the anti-viral properties of RTAM-PAP1 will be even greater than that of either RTA-PAPS1 or PAPs with even lesser general toxicity. It is further encompassed that the fusion protein encompassed herein will be effective against cancer and particularly cancer caused by viruses such as the papillomavirus. For example, HBV and HCV infection can cause liver cancer; the Kaposi Sarcoma-Associated Herpesvirus (KSHV) causing Kaposi sarcoma; Merkel Cell Polyomavirus (MCV) causing skin cancer or Merkel cell carcinoma; Human T-Cell Lymphotropic Virus Type 1 (HTLV-1) causing leukemia and lymphoma; Epstein-Barr Virus (EBV), causing Burkitt's lymphoma, Nasopharyngeal carcinoma (cancer of the upper throat), Hodgkin's and non-Hodgkin's lymphoma, T-cell lymphomas, Post-transplant lymphoproliferative disorder, or Leiomyosarcoma. In another embodiment, it is encompass that the fusion protein encompassed herein will be effective against a viral infection caused by the Japanese encephalitis virus, Herpes Simplex, Influenza virus, and/or Poliovirus.

### EXAMPLE I

### E. coil In Vivo Expression System and Rabbit Reticulate Lysate Protein Synthesis Inhibition

The two cDNA sequences coding for RTA-PAPS1 (541 amino acids) and for RTAM-PAP1 (556 amino acids including the N terminal 6-His tag) were optimized for E. coli expression and chemically synthesized by AscentGene.

The cDNA coding for RTA-PAPS1 and RTAM-PAP1 sequences described above were generated by PCR using the primers RP1-A48 (5′TTTAACTTTAAGAAGGAGATATACATATGATCTTCCCGAAACAGTACC; SEQ ID NO: 3) or RPAP1-A48 (5′TTTAACTTTAAGAAGGAGATATACATATGCACCA CCATCACCACCATA; SEQ ID NO: 4) and RPAP1-B50 (5′CAGCCGGATC TCAGTGGTGGTGCTCGAGTTAGGTAGTCTGGCAAGAACCG; SEQ ID NO: 5). Each PCR fragment was then subcloned into the E. coli pET30a expression vector (Novagene) between the NdeI and XhoI restriction endonuclease sites to generate the pET30a-RP1 and pET30a-6H-RPAP1 vectors respectively. The inserts were validated by DNA sequencing.

The above described vectors were transformed into E. coli BL21(DE3) cells (NEB) and expression of the proteins were examined from individual clones and analyzed by either Western blot using a monoclonal antibody specific to ricin A chain (ThermoFisher, RA999) or SDS gel stained with Comassie blue (ThermoFisher). Optimal conditions were determined and protein production induced in the presence of 1 mM IPTG from 1 L culture for each protein. The bacteria were then harvested by centrifugation, followed by lysing the cell pellets with 50 ml of lysis buffer (50 mM Tris-CI, 150 mM NaCl, 0.2% Triton X100 and 0.5 mM EDTA). After sonication (3×2 min), the soluble lysates were recovered by centrifugation at 35K rpm for 40 min. The insoluble pellets were further extracted with 40 ml of 6M Urea and the inclusion bodies (IB) were recovered by centrifugation at 16K rpm for 20 min. Clarified IB were then dissolved with 20 ml of buffer 8b (proprietary formulation of AscentGene). The soluble proteins were then recovered by centrifugation (please contact the authors for more details).

Ricin-PAPS1 proteins were purified by gel filtration column (Superdex 200 from GE Healthcare) under denaturing condition (6M Urea). Peak fractions were pooled and powder Guanidine was added to a concentration of 5M for complete denaturing. Denatured Ricin-PAPS1 was then added dropwise to the refolding buffer (50 mM Tris-CI, pH 8.1, 0.4M L-Arginine, 0.5 mM oxidized glutathione and 5 mM reduced glutathione) for refolding. The solution was stirred at room temperature for 10 min before allowing the refolding reaction to be further carried out at 4° C. for >20 hrs. Clarified and refolded Ricin-PAPS1 proteins were then concentrated before going through the endotoxin removal process and the ammonium sulfate precipitation step. The resulting mixture was dialyzed in the formulation buffer containing 20 mM HEPES-Na, pH 7.9, 20% glycerol, 100 mM NaCl, 2.5 mM tris(2-carboxyethyl)phosphine (TCEP) and 1 mM EDTA.

The purification of the native RTAM-PAP1 from soluble lysate was achieved by affinity versus His-tag on Ni-sepharose column (GE Healthcare). After extensive washes with the lysis buffer, loosely bound proteins were eluted with the lysis buffer containing 40 mM Imidazole (140). RTAM-PAP1 proteins were eluted with the elution buffer (20 mM Tris-CI, pH 7.9, 100 mM NaCl, 1 mM EDTA and 300 mM Imidazole). A second purification step using Hydroxylapatite column (GE Healthcare) was used to further separate RTAM-PAP1 from co-purified host proteins. A third purification step, gel filtration on a fast protein liquid chromatography (FPLC) column of Superose 12 (GE Healthcare), was necessary to completely get rid of degraded and/or premature protein products. The resulting mixture was dialyzed in the formulation buffer containing 20 mM HEPES-Na, pH 7.9, 200 mM NaCl, 0.2 mM CaCl₂ and 0.5 mM EDTA.

The inhibitory activities of RTA-PAPS1 and RTAM-PAP1 were tested by using the Rabbit Reticulate Lysate TnT® Quick Coupled Transcription/Translation System and the Luciferase Assay System (Promega). Briefly, each transcription/translation reaction was performed according to the instructions for use (IFU) in the presence of a T7 Luciferase reporter DNA, and the Luciferase expression level was determined with a Wallac Microplate Reader. Transcription/translation runs were done twice with and without addition of five different concentrations of RTA-PAPS1 and RTAM-PAP1 in order to determine the inhibitory effect of the proteins. RTA-PAPS1 and RTAM-PAP1 concentrations were adjusted by taking sample purity into consideration.

### EXAMPLE II

### Anti-HBV Assay

The anti-HBV assay was performed as previously described (Min et al., 2017, Journal of Medicinal Chemistry, 60: 6220-6238) with the modification of using HepAD38 cells by ImQuest BioSciences. ImQuest BioSciences developed a multi-marker screening assay utilizing the HepAD38 cells to detect proteins, RNA, and DNA intermediates characteristic of HBV replication. The HepAD38 cells are derived from HepG2 stably transfected with a single cDNA copy of hepatitis B virus pregenomic RNA, in which HBV replication is regulated by tetracycline. Briefly, HepAD38 cells were plated in 96-well flat bottom plates at 1.5×10⁴ cells/well in Dulbecco's modified Eagle's medium supplemented with 2% FBS, 380 μg/mL G418, 2.0 mM L-glutamine, 100 units/mL penicillin, 100 μg/mL streptomycin, and 0.1 mM nonessential amino acids (ThermoFisher). After 24 h, six tenfold serial dilutions of RTA-PAPS1 prepared in the same medium were added in triplicate. Lamivudine (3TC from Sigma Aldrich) was used as the positive control, while media alone was added to cells as a negative control (virus control, VC). Three days later, the culture medium was replaced with fresh medium containing the appropriately diluted RTA-PAPS1. Six days following the initial administration of RTA-PAPS1, the cell culture supernatant was collected, diluted in qPCR dilution buffer, and then used in a real-time quantitative qPCR assay using a Bio-Rad CFX384 Touch Real-Time PCR Detection System. The HBV DNA copy number in each sample was interpolated from the standard curve by the supporting software. A tetrazolium dye uptake assay (ThermoFisher) was then employed to measure cell viability, which was used to calculate cytotoxic concentration (TC₅₀).

### EXAMPLE III

### Protein Design Optimization

The molecular profile of the protein was determined using the Protparam tool of ExPASy, and the solubility of these proteins was determined using Predict Protein. The presence of disulfide bonds was determined using the DiANNA 1.1 webserver. Functional effects of point mutations were determined using SNAP2 of Predict Protein.

The structure of the protein was predicted by fold recognition methodology using the I-TASSER and Phyre2 prediction servers. The determined protein structures were then validated by Verify 3D. The quality of the structure was determined using the QMEAN6 program of the SWISS-MODEL workspace.

Three major changes were made to RTA-PAPS1 in order to increase its solubility, its efficacy against infected cells and to further reduce its toxicity.

Firstly, two point mutations, as predicted by SNAP2 of Predict Protein to have the least effect on function, were introduced into the RTA moiety to replace the Cysteine (Cys) residues with Alanine residues in order to completely avoid unwanted disulfide bond formation at position 171 and 259 (C171A and C259A) to create RTA mutant (RTAM).

Secondly, the natural semi-flexible linker previously used was replaced with a newly designed soluble flexible G rich linker with a rigid CASP2 recognition site (GGGGSDVADI(GGGGS)₂) to allow better autonomous function of each moiety with minimal steric hindrance and to further enhance the chimeric protein's ability to induce cell apoptosis.

Thirdly, a different variant than PAPS1 was used, PAP1, retrieved from National Centre for Biotechnology Information database (NCBI) with access number P10297.2 (SEQ ID NO: 6) in order to further enhance activity against HBV and further reduce toxicity of the chimeric protein.

Lastly, a 6-His tag was added at the N terminal of the protein RTAM-PAP1 in order to minimize effect on structure and function and to increase native protein recovery from E. coli production.

While the present disclosure has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

1. An anti-viral fusion protein comprising the structure:
X-Y-Z
wherein X is a full length Ricin A chain (RTA) or a variant thereof, Y is absent or a linker and Z is a full length Pokeweed antiviral protein (PAP) or a variant thereof.

2. The anti-viral fusion protein of claim 1, wherein Z is the Pokeweed Antiviral Protein from Leaves (PAP1).

3. The anti-viral fusion protein of claim 2, wherein PAP1 comprises amino acids 296-556 of SEQ ID NO: 2.

4. The anti-viral fusion protein of claim 1, wherein the linker is chemical linker or a polylinker.

5. The anti-viral fusion protein of claim 1, wherein the linker is a flexible linker.

6. The anti-viral fusion protein of claim 5, wherein the flexible linker comprises amino acids 275-295 of SEQ ID NO: 2.

7. The anti-viral fusion protein of claim 1, wherein X is a mutant of RTA (RTAM).

8. The anti-viral fusion protein of claim 7, wherein RTAM comprises amino acids 8-274 of SEQ ID NO: 2.

9. The anti-viral fusion protein of claim 1, comprising the amino acid sequence of SEQ ID NO: 1.

10. The anti-viral fusion protein of claim 1, comprising the amino acid sequence of SEQ ID NO: 2.

11. The anti-viral fusion protein of claim 1, for treating a viral infection.

12. The anti-viral fusion protein of claim 11, wherein the viral infection is from the Hepatitis B virus (HBV), Hepatitis C virus (HCV), Kaposi Sarcoma-Associated Herpesvirus (KSHV), Merkel Cell Polyomavirus (MCV). Human T-Cell Lymphotropic Virus Type 1 (HTLV-1), Epstein-Barr Virus (EBV), human immunodeficiency virus-1 (HIV-1), Zika virus, Japanese encephalitis virus, Herpes Simplex, Poliovirus, Influenza virus, coronavirus or papillomavirus.

13. The anti-viral fusion protein of claim 11, wherein the viral infection causes liver cancer, Kaposi sarcoma, skin cancer, Merkel cell carcinoma, leukemia, lymphoma, Burkitt's lymphoma, Nasopharyngeal carcinoma, Hodgkin's lymphoma, non-Hodgkin's lymphoma, T-cell lymphomas, Post-transplant lymphoproliferative disorder, respiratory disease or Leiomyosarcoma.

14-15. (canceled)

16. The anti-viral protein of claim 1, wherein said fusion protein is active against plant, animal or human pathogens.

17. (canceled)

18. A composition comprising the fusion protein of claim 1 and a carrier.

19. A method of treating a viral infection in a patient comprising administering to said patient a fusion protein of claim 1.

20. The method of claim 19, wherein the viral infection is from the Hepatitis B virus (HBV), Hepatitis C virus (HCV), Kaposi Sarcoma-Associated Herpesvirus (KSHV), Merkel Cell Polyomavirus (MCV). Human T-Cell Lymphotropic Virus Type 1 (HTLV-1), Epstein-Barr Virus (EBV), human immunodeficiency virus-1 (HIV-1), Zika virus, Influenza virus, coronavirus or papillomavirus.

21. The method of claim 19, wherein the viral infection causes liver cancer, Kaposi sarcoma, skin cancer, Merkel cell carcinoma, leukemia, lymphoma, Burkitt's lymphoma, Nasopharyngeal carcinoma, Hodgkin's lymphoma, non-Hodgkin's lymphoma, T-cell lymphomas, Post-transplant lymphoproliferative disorder, respiratory disease or Leiomyosarcoma.

22-23. (canceled)

24. The method of any one of claims 19-23, wherein said fusion protein is active against plant, animal or human pathogens.

25-30. (canceled)
